(19) United States
(12) Patent Application Publication
(10) Pub. No.: US 2015/0080615 A1
(43) Pub. Date: Mar. 19, 2015

(54) HIGH TEMPERATURE ESTER HYDROLYSIS OPERATING AT HIGH ESTER TO WATER RATIOS

(71) Applicant: Butamax Advanced Biofuels LLC, Wilmington, DE (US)

(72) Inventors: STUART B. FERGUSSON, Kingston (CA); LARIN GODFROY, Napanee (CA); MICHAEL CHARLES GRADY, Oaklyn, NJ (US); DAVID F. KRAYNIE, Chadds Ford, PA (US); WILLIAM D. PARTEN, Wilmington, DE (US); STEPHEN M. TIERI, Wilmington, DE (US)

(21) Appl. No.: 14/484,676

(22) Filed: Sep. 12, 2014

Related U.S. Application Data
(60) Provisional application No. 61/877,326, filed on Sep. 13, 2013.

(51) Int. Cl. C07C 29/14

(52) U.S. Cl. CPC C07C 29/14
USPC 568/877

(57) ABSTRACT

A process for recovering alcohol from a fatty acid and/or diacid alcohol ester using water in an equal or lesser amount than oil on a mass basis. The process uses multiple reactors with separation of the alcohol product of hydrolysis between successive reactors. The use of low amounts of water allows recovery of the alcohol with a lower evaporation requirement, thus making a more energy efficient process.

### CROSS-REFERENCE TO RELATED APPLICATION

This application claims benefit of priority from U.S. Provisional Application No. 61/877,326, filed Sep. 13, 2013, which is hereby incorporated by reference in its entirety.

### FIELD OF THE INVENTION

The invention relates to product recovery. More specifically, the invention relates to recovery of alcohols from carboxylic acid esters using a low amount of water, with high temperature and pressure, and more than one reactor in succession.

### BACKGROUND OF THE INVENTION

Alcohols have a variety of applications in industry and science such as a beverage (i.e., ethanol), fuel, reagents, solvents, and antiseptics. For example, butanol is an alcohol that is an important industrial chemical and drop-in fuel component with a variety of applications including use as a renewable fuel additive, as a feedstock chemical in the plastics industry, and as a food-grade extractant in the food and flavor industry. Accordingly, there is a high demand for alcohols such as butanol, as well as for efficient and environmentally-friendly production methods.

Production of alcohol utilizing fermentation by microorganisms is one such environmentally-friendly production method. In the production of butanol, in particular, some microorganisms that produce butanol in high yields also have low butanol toxicity thresholds. One method of mitigating the toxic effect of an alcohol, such as butanol, on the production microorganism is to esterify the alcohol with carboxylic acid, such as fatty acid, thereby converting the alcohol to a compound with reduced toxicity. This process is disclosed in U.S. Patent Publication No. 20120156738. The esterified product must then be hydrolyzed to recover the alcohol as a product.

U.S. Pat. No. 6,646,146 discloses a process for hydrolyzing a fatty acid alcohol ester in liquid phase by contact with steam in the presence of a metal catalyst capable of forming a soap with a large hydration shell at pressure between 43.5-290 psi.

EP 1352891 discloses a process for hydrolyzing a fatty acid ester in liquid phase by contact with water in the presence of a solid acid catalyst.

There remains a need for a process for recovering alcohol from carboxylic acid alcohol esters that is effective and that also requires reduced energy input so that it is commercially viable.

### SUMMARY OF THE INVENTION

The invention provides a process for recovering alcohol from fatty acid and diacid alcohol esters.

Accordingly, the invention provides process for recovering alcohol from one or more fatty acid alcohol esters and/or one or more diacid alcohol esters comprising: (a) contacting one or more fatty acid and/or diacid alcohol esters in an oil phase with water producing a first reaction mixture in a first reactor at a temperature between 150° C. and 350° C. and a pressure high enough to keep the reaction in the liquid phase, using a mass ratio of oil to water that is at least about 1:1, wherein a portion of the alcohol is released from the fatty acid and/or diacid ester; (b) separating by boiling point difference a first alcohol and steam stream from the first reaction mixture in the first reactor, leaving a first process oil phase containing remaining fatty acid and/or diacid alcohol esters; (c) passing the first process oil phase containing remaining fatty acid and/or diacid alcohol esters to a second reactor and contacting the first process oil phase with water producing a second reaction mixture at a temperature between 150° C. and 350° C. and a pressure high enough to keep the reaction in the liquid phase, using a mass ratio of oil to water that is at least about 1:1, wherein a portion of the alcohol is released from the fatty acid and/or diacid alcohol ester; (d) separating by boiling point difference a second alcohol and steam stream from the second reaction mass in the second reactor, leaving a second process oil phase containing remaining fatty acid and/or diacid alcohol esters; and (e) recovering alcohol from the first and second alcohol and steam streams.

### BRIEF DESCRIPTION OF THE DRAWINGS

FIG. 1 is a schematic drawing showing process flows for one reactor recovering alcohol from fatty acid and/or diacid alcohol esters.

FIG. 2 is a schematic drawing showing an ester hydrolysis reactor train.

### DETAILED DESCRIPTION

The following definitions and abbreviations are to be used for the interpretation of the claims and the specification.

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

The term “invention” or “present invention” as used herein is a non-limiting term and is not intended to refer to any single embodiment of the particular invention but encompasses all possible embodiments as described in the specification and the claims.

As used herein, the term “about” modifying the quantity of an ingredient or reactant of the invention employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. In one embodiment, the term “about” means within 10% of the reported numerical value, preferably within 5% of the reported numerical value.

The term “carboxylic acid” as used herein refers to any organic compound with the general chemical formula —COOH in which a carbon atom is bonded to an oxygen atom by a double bond to make a carbonyl group (—C═O) and to a hydroxyl group (—OH) by a single bond. A carboxylic acid may be in the form of the protonated carboxylic acid, in the form of a salt of a carboxylic acid (e.g., an ammonium, sodium, or potassium salt), or as a mixture of protonated carboxylic acid and salt of a carboxylic acid. The term carboxylic acid may describe a single chemical species (e.g., oleic acid) or a mixture of carboxylic acids as can be produced, for example, by the hydrolysis of biomass-derived fatty acid esters or triglycerides, diglycerides, monoglycerides, and phospholipids. “Diacid” refers to dicarboxylic acids.

The term “fatty acid” as used herein refers to a carboxylic acid (e.g., aliphatic monocarboxylic acid) having C₄ to C₂₈ carbon atoms (most commonly C₁₂ to C₂₄ carbon atoms), which is either saturated or unsaturated. Fatty acids may also be branched or unbranched. Fatty acids may be derived from, or contained in esterified form, in an animal or vegetable fat, oil, or wax. Fatty acids may occur naturally in the form of glycerides in fats and fatty oils or may be obtained by hydrolysis of fats or by synthesis. The term fatty acid may describe a single chemical species or a mixture of fatty acids. In addition, the term fatty acid also encompasses free fatty acids.

“Native oil” as used herein refers to lipids obtained from plants (e.g., biomass) or animals. “Plant-derived oil” as used herein refers to lipids obtained from plants in particular. From time to time, “lipids” may be used synonymously with “oil” and “acyl glycerides.” Native oils include, but are not limited to, tallow, corn, canola, capric/caprylic triglycerides, castor, coconut, cottonseed, fish, jojoba, lard, linseed, neetsfoot, oiticica, palm, peanut, rapeseed, rice, safflower, soya, sunflower, tung, jatropha, and vegetable oil blends.

As used herein, the term “solid acid catalyst” refers to any solid material containing Brönsted and/or Lewis acid sites, and which is substantially undissolved by the reaction medium to which it is added under ambient conditions.

As used herein, the term “alcohol” refers to any alcohol that can be produced in a fermentation process. Alcohols include, but are not limited to, C₂ to C₂₀ alcohols. The alcohol can, for example, be a diol. The alcohol can, for example, be a C₁ to C₈ alkyl alcohol. Examples of C₁ to C₈ alkyl alcohols include, but are not limited to, ethanol, propanol, and butanol (e.g., 1-butanol, 2-butanol, and isobutanol).

The invention relates to a process for recovering alcohol from fatty acid and/or diacid alcohol esters. In the present process, water is used in an equal or lesser amount than oil on a mass basis. This is made possible by using multiple reactors with separation of the alcohol product of hydrolysis between successive reactors. The use of low amounts of water allows recovery of the alcohol with a lower evaporation requirement, thus making a more energy efficient process.

### Process Description

Fatty acid and/or diacid alcohol esters are contacted and mixed with water forming a reaction mixture and held at reaction temperature in a volume sufficient to provide adequate residence time to affect the conversion of a portion of the ester of fatty acid or diacid to alcohol plus fatty acid or diacid. The alcohol esters may be fatty acid esters, diacid esters, or a mixture of fatty acid and diacid alcohol esters. A preparation of fatty acid and/or diacid alcohol esters may include unreacted fatty acids and/or diacids. Oil phase herein refers to a mixture of fatty acid and/or diacid alcohol esters, and may include unreacted fatty acids and/or diacids as well as fatty acids and/or diacids that result from previously hydrolyzed alcohol esters. The oil phase and water mixture has a mass ratio of oil to water that is at least about 1:1. The ratio may be at least about 1.5:1, 2:1, 3:1, 4:1, 5:1, or greater. With less water, less energy is required for distillation during alcohol recovery as compared to the energy required to recover alcohol in a stream from a process where the mass ratio of oil to water is less than 1:1.

The reaction temperature is between about 150° C. and about 350° C. Pressure in a reactor containing the oil and water mixture is high enough to keep the reaction in the liquid phase, as will be known by one skilled in the art based on the known vapor pressures of water and product alcohol and the fatty acid and/or diacid alcohol esters and fatty acids and/or diacids of the reaction mixture. Typically the pressure may be about 10-50 psig (69-345 kilopascal) above the vapor pressure of the most volatile compounds in the reaction mixture at the reaction temperature. For example, in one embodiment the temperature is at least about 190° C. and the pressure is at least about 210 psig (1.4 megapascal). In another embodiment the temperature is at least about 225° C. and the pressure is at least about 350 psig (2.4 megapascal). For example, where the isobutyl ester of soybean oil fatty acids is the starting material, a temperature of 260° C. and a pressure of 800 psig (5.5 megapascal) is used herein in Example 2. By way of another example, where the isobutyl ester of corn oil fatty acids is the starting material, a temperature of 260° C. and a pressure of 735 psig is used herein in Example 7.

The volume of the reactor and the flow rate of the oil phase into the reactor may be adjusted, as known to one skilled in the art, to vary the residence time of the oil phase in the reactor where it is in contact with water. In various embodiments the residence time in a reactor is less than four hours, three hours, two hours, or one hour. In one embodiment the residence time in a reactor is thirty minutes or less. The present process allows low residence time, thereby allowing the reactor to be smaller and less costly. The residence time is a sufficient amount of time for conversion of a portion of the fatty acid and/or diacid alcohol ester to alcohol and fatty acid and/or diacid.

The reactor may be in either a vertical or a horizontal orientation. For example, the reactor may be a pipe reactor having a feed end and a removal end, or a vertical reactor with bottom feed and top removal.

In one embodiment the oil phase is fed to the bottom of the reactor after being pre-heated to reaction temperature just prior to entering the reactor to avoid unnecessary side reactions brought on by high temperature and/or the presence of oxygen. Steam may be used to pre-heat the oil phase prior to being fed to the reactor using a heat exchanger. In another embodiment the oil phase is not pre-heated, but one or more injections of hot water or steam are injected into the reactor along the length of the reactor to heat the oil phase and the reaction mixture to temperature.

In one embodiment the water phase is pre-heated to reaction temperature before entering the reactor and fed to the top of the reactor. In other embodiments the water phase is fed as steam generated in a steam boiler of adequate pressure to achieve the temperature necessary to maintain reaction temperature.

In one embodiment the water and oil are fed together into the bottom of the reactor, with either preheating or heating in the reactor.

The oil and water phases may be mixed with agitation using static mixers and/or impellors.

The density differences between the oil and water phases allow the phases to fall and rise through the reactor thereby providing adequate mass transfer to affect the reaction. In this way an oil phase leaves the top of the reactor being a solution of the ester of fatty acid and/or diacid, fatty acid and/or diacid, alcohol and dissolved water. Some amount of entrained aqueous phase may also be present. At the same time an aqueous phase leaves the bottom of the reactor comprised mainly of water and alcohol with only a small amount of dissolved ester of fatty acid and/or diacid and fatty acid and/or diacid. Some amount of entrained oil phase may be present in the aqueous phase. Alternatively, in some embodiments the amount of water relative to oil may be kept low enough such that there is not a separate water phase leaving the bottom of the reactor.

In one embodiment the water and oil are fed together into the bottom of the reactor, with either preheating or heating in the reactor.

An alcohol and steam stream is separated by boiling point difference from the oil phase leaving the top of the reactor. Any method known to one skilled in the art may be used such as flashing or distillation. This alcohol and steam stream separated from the reactor is distilled to recover the alcohol.

The remaining oil phase is subsequently fed to another reactor operated in a similar fashion as described above where an additional portion of the fatty acid and/or diacid alcohol ester is converted to fatty acid and/or diacid, and alcohol. The process of feeding the remaining oil phase to a subsequent reactor may be repeated one or more times such that in various embodiments the total number of reactors is two, three, four, five, six or more with each successive process oil phase passing to an additional reactor. The reaction in the first reactor is a first reaction mixture, and a first alcohol and steam stream is separated from a first process oil phase containing remaining fatty acid and/or diacid alcohol esters in the first reactor. The reaction mixture in the second reactor is a second reaction mixture, and a second alcohol and steam stream is separated from a second process oil phase containing remaining fatty acid and/or diacid alcohol esters in the second reactor and so forth for the third, fourth, fifth, sixth reactors and so on when included.

An additional portion of alcohol is released from the oil phase in each reactor. The portion of alcohol released in each reactor is in an amount such that a substantial portion of the alcohol is recovered in combination from all of the reactors from the initial fatty acid and/or diacid alcohol esters. In one embodiment the alcohol and steam stream from each reactor contains at least about 25% alcohol. In other embodiments the alcohol and steam stream from each reactor contains alcohol in at least about 30%, 33%, 36%, 39%, or more. Different amounts of alcohol may be present in the alcohol and steam streams from each reactor.

By separating the water and alcohol away from the oil in a series of reactions with intermittent separations, the conversion can be driven further in less time than using a more conventional longer time and one time separation process. For example, in three reactors with a half hour of residence time in each, an oil phase was processed from a 92:8 ratio of fatty acid butyl ester:fatty acid to a ratio of 34:66. Overall, 63% conversion was achieved in 1.5 hours (see, e.g., Example 2). By way of another example, in three reactors, an oil phase was processed from a 90:10 ratio of fatty acid butyl ester:fatty acid to a 18:82 ratio of (see, e.g., Example 7). One skilled in the art can vary the parameters including temperature, pressure, residence time, and number of reactors to achieve a desired level of alcohol recovery from a fatty acid and/or diacid alcohol ester-containing oil phase.

The recovered alcohol and steam streams from the reactors may be combined for further alcohol recovery, or each may be handled separately.

In some embodiments where there is a water phase exiting the reactor, the water stream is recycled to the front of the reactor as the contacting water provided that the concentration of product alcohol is low enough in the water phase to not reverse the reaction of the ester of fatty acid and/or diacid to fatty acid and/or diacid and alcohol.

In one embodiment flashing of the water and product alcohol occurs simultaneously as the reaction proceeds. This process may be performed in a stirred tank reactor with a vent valve through which the alcohol and water can flash.

In other embodiments, a catalyst can be added to the reactor to reduce the temperature and pressure at which the reaction occurs. For example in some embodiments, in the presence of a catalyst the temperature is about 190° C. and the pressure is about 210 psig (1.45 megapascal). Catalysts that may be used include solid acid catalysts and water-tolerant Lewis acids. The solid acid catalyst is a solid acid having the thermal stability required to survive reaction conditions. Examples of suitable solid acid catalysts include without limitation the following categories: 1) heterogeneous heteropolyacids (HPAs) and their salts, 2) natural or synthetic clay minerals, such as those containing alumina and/or silica (including zeolites), 3) cation exchange resins, 4) metal oxides, 5) mixed metal oxides, 6) metal salts such as metal sulfides, metal sulfates, metal sulfonates, metal nitrates, metal phosphates, metal phosphonates, metal molybdates, metal tungstates, metal borates, and 7) combinations of any members of any of these categories. The metal components of categories 4 to 6 may be selected from elements from Groups 1 through 12 of the Periodic Table of the Elements, as well as aluminum, chromium, tin, titanium, and zirconium. Examples include, without limitation, sulfated zirconia and sulfated titania.

Suitable HPAs include compounds of the general formula XₐM_{b}O_{c}^{q-}, where X is a heteroatom such as phosphorus, silicon, boron, aluminum, germanium, titanium, zirconium, cerium, cobalt or chromium, M is at least one transition metal such as tungsten, molybdenum, niobium, vanadium, or tantalum, and q, a, b, and c are individually selected whole numbers or fractions thereof. Non-limiting examples of salts of HPAs are lithium, sodium, potassium, cesium, magnesium, barium, copper, gold and gallium, and onium salts such as ammonia. Methods for preparing HPAs are well known in the art and are described, for example, in Hutchings, et al, (Catal Today (1994) p 23). Selected HPAs are also available commercially, for example, through Sigma-Aldrich Corp. (St. Louis, Mo.). Examples of HPAs suitable for the disclosed process include, but are not limited to, tungstosilicic acid (H4[SiW12O40].xH2O), tungstophosphoric acid (H4[SiW₁₂O₄₀].xH20), tungstophosphoric acid (H₃[PW₁₂O₄₀].xH₂O), molybdophosphoric acid (H₃[PMo₁₂O₄₀].xH₂O), molybdosilicic acid (H₄[SiMo₁₂O₄₀].xH₂O), vanadotungstosilicic acid (H₄₊ₙ[SiVₙW₁₂₋ₙO₄₀].xH₂O), vanadotungstophosphoric acid (H₃₊ₙ[PVₙW₁₂₋ₙO₄₀].xH₂O), vanadomolybdophosphoric acid (H₃₊ₙ[PVₙMo₁₂₋ₙO₄₀].xH₂O), vanadomolybdosilicic acid (H₄₊ₙ[SiVₙMo₁₂₋ₙO₄₀].xH₂O), molybdotungstosilicic acid (H₄[SiMoₙW₁₂₋ₙO₄₀].xH₂O), molybdotungstophosphoric acid (H₃[PMoₙW₁₂₋ₙO₄₀].xH₂O), wherein n in the formulas is an integer from 1 to 11 and x is an integer of 1 or more.

Natural clay minerals are well known in the art and include, without limitation, kaolinite, bentonite, attapulgite, montmorillonite and zeolites.

The solid acid catalyst may be a cation exchange resin that is a sulfonic acid or carboxylic acid functionalized polymer. Suitable cation exchange resins include, but are not limited to the following: styrene-divinylbenzene copolymer-based strong cation exchange resins such as Amberlyst™ and Dowex® available from Dow Chemicals (Midland, Mich.) (for example, Dowex® Monosphere M-31, Amberlyst™ 15, Amberlyst™ 70, Amberlite™ 120); CG resins available from Resintech, Inc. (West Berlin, N.J.); Lewatit resins such as MonoPlus™ S 100H available from Sybron Chemicals Inc. (Birmingham, N.J.); fluorinated sulfonic acid polymers (these acids are partially or totally fluorinated hydrocarbon polymers containing pendant sulfonic acid groups, which may be partially or totally converted to the salt form) such as Nafion® perfluorinated sulfonic acid polymer, Nafion® CR carboxylic acid, Nafion® Super Acid Catalyst (a bead-form strongly acidic resin which is a copolymer of tetrafluoroethylene and perfluoro-3,6-dioxa-4-methyl-7-octene sulfonyl fluoride, converted to either the proton (H⁺), or the metal salt form available from DuPont Company (Wilmington, Del.).

The solid acid catalyst may be supported, wherein the support can be any solid substance that is inert under the reaction conditions including, but not limited to, oxides such as silica, alumina, titania, sulfated titania, and compounds thereof and combinations thereof; barium sulfate; calcium carbonate; zirconia; carbons, particularly acid washed carbon; and combinations thereof. Acid washed carbon is a carbon that has been washed with an acid, such as nitric acid, sulfuric acid or acetic acid, to remove impurities. The support can be in the form of powder, granules, pellets, or the like. The supported acid catalyst can be prepared by depositing the acid catalyst on the support by any number of methods well known to those skilled in the art of catalysis, such as spraying, soaking or physical mixing, followed by drying, calcination, and if necessary, activation through methods such as reduction or oxidation. The loading of the at least one acid catalyst on the at least one support is typically in the range of 0.1-20 weight % based on the combined weights of the at least one acid catalyst and the at least one support. Certain acid catalysts perform better at low loadings such as 0.1-5%, whereas other acid catalysts are more likely to be useful at higher loadings such as 10-20%. Examples of supported solid acid catalysts include, but are not limited to, phosphoric acid on silica, Nafion® perfluorinated sulfonic acid polymer on silica (SiO₂), HPAs on silica, sulfated zirconia, and sulfated titania. In the case of Nafion® on silica, a loading of 12.5% is typical of commercial examples.

Alternatively, the acid catalyst is an unsupported catalyst having 100% acid catalyst with no support such as, pure zeolites and acidic ion exchange resins.

Zeolites suitable for use herein can be generally represented by the following formula M_{2/n}O.Al₂O₃.xSiO₂.yH₂O wherein M is a cation of valence n, x is greater than or equal to about 2, and y is a number determined by the porosity and the hydration state of the zeolite, generally from about 2 to about 8. In naturally occurring zeolites, M is principally represented by Na, Ca, K, Mg and Ba in proportions usually reflecting their approximate geochemical abundance. The cations M are loosely bound to the structure and can frequently be completely or partially replaced with other cations by conventional ion exchange. Zeolite pore dimensions, the presence of ions near the port, Si/AL ratio, and hydrophobicity/hydrophilicity are factors contributing to catalytic properties.

Other molecular sieves which have structures similar to zeolites may be used as catalysts in the present process. These molecular sieves contain other elements in place of aluminum and silicon. Examples of such molecular sieves include without limitation Ti-Beta, B-Beta, Sn-Beta, Zn-Beta, and Ga-Beta silicates.

Alternatively, catalysts used in the present process may be water tolerant Lewis acids, such as a metal salt catalyst of general formula MA_{X} wherein A is a non-coordinating or weakly coordinating anion and M is a Group IIIB, rare earth or lanthanide, actinide or Group IVB cation with x being the valence of M. By the term “water-tolerant” it is meant that the Lewis acid is not hydrolyzed by water. By the term “non-coordinating or weakly coordinating anion” it is meant that the anion is not bound to the metal in an aqueous solution. Examples of a non-coordinating or weakly coordinating anion in the present process are trifluoromethane sulfonate, also known as triflate ([CF₃SO₃]⁻), hexafluorophosphate ([PF₆]⁻), [Al[OC(CF₃)₃]₄]⁻, tetrafluoroborate ([BF₄]⁻), perchlorate ([ClO₄]⁻), teflate ([TeOF₅]⁻), BArF ([B(ArHₓF_{y})₄]⁻, where Ar is an aryl and x+y=5, e.g., [B(C₆F₅)₄]⁻, tosylate ([CH₃C₆H₄SO₃]⁻, mesylate ([CH₃SO₃]⁻) and antimonyhexafluoride ([SbF₆]⁻). Further examples of non-coordinating or weakly coordinating anions are found in “The Search for Larger and More Weakly Coordinating Anions”, Steven H. Strauss, Chem. Rev. vol. 93, p. 927-942 (1993) and “Structure and Characterization of Cl₃⁺[Al{OC(CF₃)₃}]⁻; Lewis Acidities of CX₃⁺ and BX₃”, Ingo Krossing et al., Angew. Chem. Int. Ed., vol. 42, p. 1531-1534 (2003), which are incorporated by reference.

Examples of Group IIIB metals are scandium and yttrium. An example of a Group IVB metal is hafnium. Examples of rare earth or lanthanide cation are lanthanum, europium and ytterbium. Examples of water tolerant Lewis acids are scandium triflate [Sc(CF₃SO₃)₃], europium triflate [Eu(CF₃SO₃)₃], hafnium triflate [Hf(CF₃SO₃)₄], yttrium triflate [Y(CF₃SO₃)₃], lanthanum triflate [La(CF₃SO₃)₃] and ytterbium triflate [Yb(CF₃SO₃)₃]. Many of these water tolerant Lewis acids are commercially available or can be synthesized by methods known in the art. The catalysts used can be homogeneous, i.e., liquid phase. The catalysts can be heterogenized using procedures known in the art. These procedures include the use of ion exchange resins, microencapsulation and binding to a metal oxide surface.

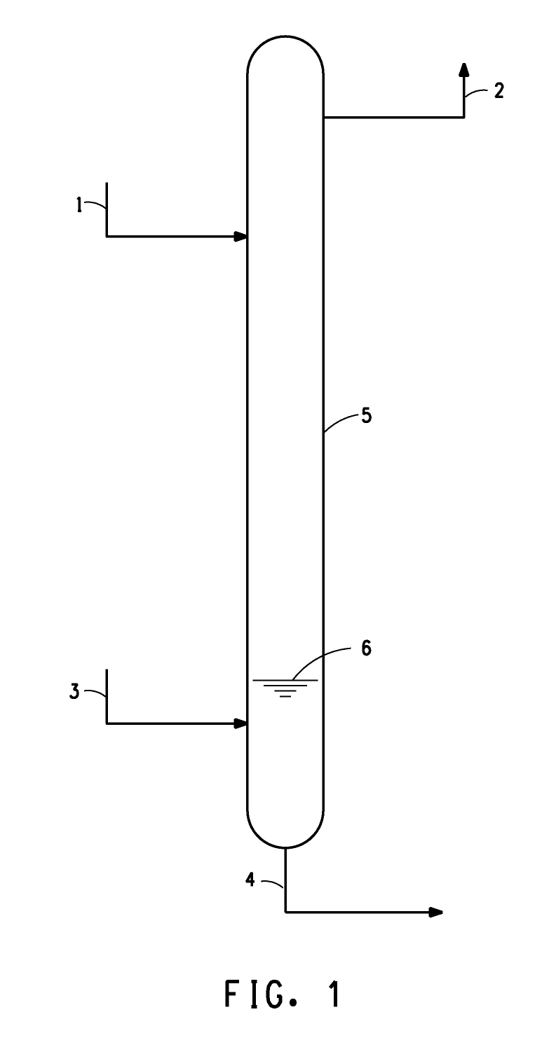

FIG. 1 shows a schematic diagram of one embodiment of a reactor unit of the present process. Water (1) enters the upper portion of a vertical reactor (5) while fatty acid and/or diacid alcohol esters (3) enter the lower portion of the reactor. An oil/water interface (6) forms in the reactor. A flow out of the upper portion of the reactor is a solution of the ester of fatty acid and/or diacid, fatty acid and/or diacid, alcohol and dissolved water.

The reactor may be made of any material that can withstand the temperature, pressure and corrosivity of the raw materials required for the reaction. For example, the reactor may be made of 304 SS, 316SS, Hastelloy, schedule 80 316SS pipe.

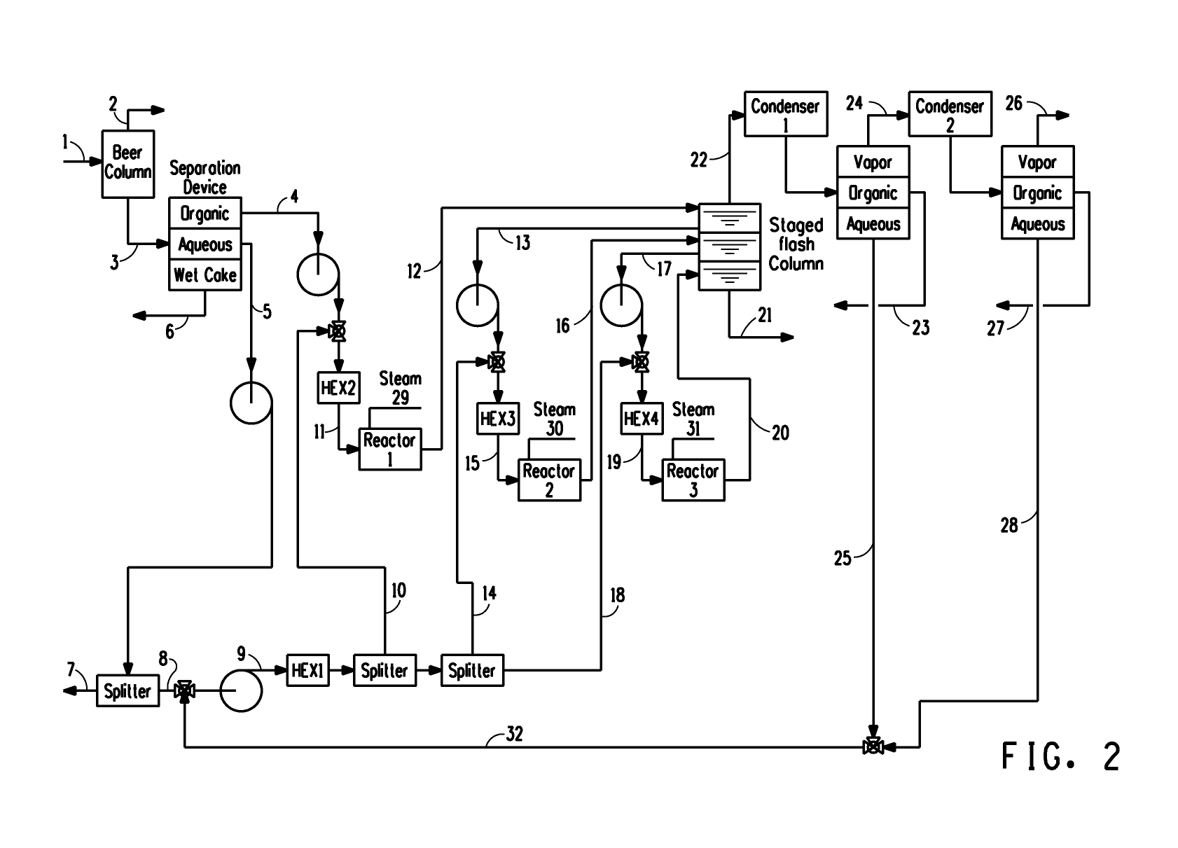

FIG. 2 shows a schematic diagram of an embodiment of an ester hydrolysis reactor train. In FIG. 2, after completion of the fermentation, Stream 1 comprising a mixture of aqueous fermentation broth containing product alcohol and an organic alcohol ester of fatty acid and fatty acid stream are fed to the process beer column. In the beer column, distillation produces Stream 2 comprising the product alcohol and water, which is taken overhead to be condensed and further purified, and Stream 3 comprising the remaining aqueous fermentation broth mixture. Stream 3 is sent to a solid/liquid/liquid separation device, which can be a centrifuge, a decanter, or some similar device. The separation device produces organic phase Stream 4, which comprises the alcohol ester of fatty acid and the fatty acid; aqueous phase Stream 5, which comprises fermentation broth mostly devoid of product alcohol; and wet cake Stream 6, which comprises the insoluble mash solids left over after fermentation. Stream 4 is pumped and heated to or near to reaction temperature and fed to the first reactor in a series of reactors. A portion of Stream 5 is split and returns to the process as Stream 7, where it can be returned to the front of the process as backset or evaporated and returned to the front of the process. The remaining portion of Stream 5, Stream 8, is mixed with Stream 32, which is aqueous water returning from the ester hydrolysis process, to form Stream 9. Stream 9 is heated to or near to reaction temperature through heat exchanger 1 (HEX1). In some embodiments, Stream 32 or Streams 25 and 28, which can make up Stream 32, can be distilled separately or added back to Stream 1 to remove the product alcohol remaining in Stream 22 before mixing with Stream 8. A portion of Stream 9, Stream 10, is mixed with Stream 4 and reheated to or near to reaction temperature through heat exchanger 2 (HEX2) and fed to Reactor 1 as Stream 11. Additional steam, Stream 29, can be injected to Reactor 1 to maintain temperature along the reactor profile. Stream 11 is subject to ester hydrolysis in Reactor 1 and the effluent, Stream 12, which comprises a mixture of the organic alcohol ester of fatty acid, fatty acid, water and alcohol are fed to an upper tray of the Staged Flash Column where the water and alcohol are flashed and separated from the liquid stream. The remaining alcohol ester of fatty acid and fatty acid in Stream 13 are pumped back and mixed with Stream 14, which is a portion of Stream 9. The two streams are heated via heat exchanger 3 (HEX3) and fed as Stream 15 to Reactor 2 where additional ester hydrolysis occurs. Additional steam, Stream 30, can be injected into Reactor 2 as needed to maintain temperature throughout the reactor. The effluent from Reactor 2, Stream 16, is fed to the flash column and the product alcohol and water present in Stream 16 is flashed into the vapor phase. The remaining liquid, Stream 17, is fed back to the next ester hydrolysis reactor along with the remaining water, Stream 18, where the mixed stream is heated to or near to reaction temperature via heat exchanger 4 (HEX4). The heated stream, Stream 19, is fed to Reactor 3. Additional steam, Stream 31, can be added to Reactor 3 to maintain temperature throughout the reactor. The effluent of Reactor 3, Stream 20, is fed to the flash column where the product alcohol and water are also flashed from the ester of fatty acid and fatty acid mixture. In certain embodiments, additional reactors can be added in series to the reactor train for further conversion of the alcohol ester of fatty acid to fatty acid and product alcohol as needed to obtain the objectives of the process. Stream 21 comprises the fatty acid and alcohol ester of fatty acid stream leaving the bottom of the flash column and represents a majority of the final product from ester hydrolysis. The vapor leaving the flash column, Stream 22, is partially condensed in Condenser 1 under conditions that generate an aqueous phase, Stream 25, an organic phase, Stream 23, and a vapor stream, Stream 24. Stream 23 is primarily fatty acid and the alcohol ester of fatty acid, which can be returned to the fermentation along with Stream 21. Stream 25 is primarily water and can be returned to the ester hydrolysis units to hydrolyze the alcohol ester of fatty acid into fatty acid and alcohol. The vapor stream, Stream 24, is condensed in Condenser 2, under conditions that generate a vapor stream, Stream 26, an aqueous phase, Stream 28 and an organic phase, Stream 27. Stream 26 can be sent to a scrubber system that removes any condensable liquids. Stream 27 contains mostly product alcohol with some water and can be sent to distillation for additional purification. Stream 28 is mostly water and some product alcohol and can be returned to the ester hydrolysis units to hydrolyze the alcohol ester of fatty acid into fatty acid and alcohol. As with Stream 25, Stream 28 can be distilled to remove the product alcohol before returning it to the ester hydrolysis reactor. In certain embodiments some of the fatty acid in Stream 21 can be recycled with Stream 4 to reduce the concentration of the alcohol ester of fatty acid in the feed stream to the ester hydrolysis units to reduce the number of units required to reach the final fatty acid content required in Stream 21. The process can be energy intergrated by altering the temperatures and pressures of the various streams leaving the different unit operations to save on energy costs.

### Alcohol Esters

Fatty acid and/or diacid alcohol esters produced by any means may be used as the oil phase in the present process. In various embodiments the esters may include alcohols containing a number of carbons up to 20, including 2, 3, 4, 5, 10, 15, or 20 carbons. Typically the number of carbons is from 4 to 20, since alcohols with a number of carbons less than four would require very high pressures based on their vapor pressures. For example, the alcohol may be butanol, such as isobutanol. The alcohol may be a diol having the described number of carbons. For example, the alcohol may be a diol such as 1,2-ethanediol, 1,2-propanediol, 1,3-propanediol, 1,4-butanediol, and 2,3-butanediol.

Fatty acid of the esters may be from any source. The fatty acid may be in a native oil such as corn oil fatty acid, soya oil fatty acid, peanut oil fatty acid, canola oil fatty acid, sunflower oil fatty acid, or a mixture of fatty acids from different types of oil. The fatty acid may be any that has a vapor pressure compatible with reaction conditions such that the fatty acid alcohol ester is in liquid phase under the reaction conditions. The fatty acids may be C₄ to C₂₈ fatty acids. More typically the fatty acids are C₁₀ to C₂₀ fatty acids, and even more useful are C₁₂ to C₁₈ fatty acids. Typically the fatty acid in a preparation of fatty acid alcohol esters will be a mixture of different types of fatty acids.

The fatty acid alcohol esters of the oil phase of the present process may be a single compound, or it may be a mixture of compounds. The mixture may include one or more alcohol in the esters, and/or one or more fatty acid in the esters.

Fatty acid alcohol esters used in the present process may be made using any method. For example, U.S. Patent Publication No. 20120156738, which is incorporated herein by reference, discloses production of carboxylic acid alcohol esters using a catalyst where the alcohol may be produced in a fermentation process and the catalyst may be an enzyme such as esterase, lipase, phospholipase, or lysophospholipase. For example, U.S. Patent Publication No. 20120322117, which is incorporated herein by reference, discloses similarly producing carboxylic acid diol esters.

Diacid of the diacid alcohol esters may be from any source. Any diacid may be used that has a vapor pressure compatible with reaction conditions such that the diacid alcohol ester is in liquid phase under the reaction conditions. Typically the diacid is a C₁₂ to C₁₈ diacid including, for example, C₁₂, C₁₆, C_{18:1}, and C_{18:2}. Diacid alcohol esters may be made by any method known to one skilled in the art. The diacid alcohol esters of the oil phase of the present process may be a single compound, or it may be a mixture of compounds. The mixture may include one or more alcohol in the esters, and/or one or more fatty acid in the esters.

In addition, the oil phase may contain both fatty acid alcohol esters and diacid alcohol esters.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations is as follows: “hr” means hour(s), “min” means minute(s), “sec” means second(s), “d” means day(s), “L” means liter(s), “mL” means milliliter(s), “μL” means microliter(s), “kg” means kilograms, “g” means grams, “mg” means milligrams, “g/L” means grams per liter, “mM” means millimolar, “nm” means nanometer(s), “mm” means millimeter.

### General Methods

### Oil Product Analysis

Oil product samples taken from top of the hydrolyser were diluted approximately 50 percent with iso-propanol (IPA), in-situ, in order to homogenize the oil and water phases. They were further diluted and methyl palmitate was added as an internal standard prior to being injected to a GC column (FFAP, Free Fatty Acid and Phenol column 20 meter column. 0.18 mm ID, 0.36 micrometer film thickness). The method was run on a 430 Bruker GC instrument with a 60 to 250° C. temperature ramp at 25 C/min and a 12.4 minute hold time. A glass fritted injector was used with a 1 microliter injection and a 25:1 split. The carrier gas was helium at 2 ml/min. Both direct Internal Standard (ISTD) calibration and Gas Chromatography area percent were applied to the sample analysis so that the results could be compared. Water is not measured in GC area percent so it is easier to see relative ratios in the oil phase only.

The precision of the analysis on an individual component is estimated to be 1.2 percent at the 95 percent confidence limit with direct analysis.

### Water Product Analysis

Water product samples taken from the bottom of hydrolyser visually look murky, cloudy and milky i.e. appearing to be an emulsion of oil and water. They usually have an oil layer on the top. These samples were homogenized with the addition of at least 50 percent isopropanol. An aliquot of the homogenized sample was further diluted ˜48 times overall in isopropanol with 2-pentanol used as the internal standard. This internal standard was used as it eluted close to iso-butanol and had exceptional precision characteristics.

The samples were analyzed by Gas Chromatography-Flame Ionization Detection, and both iso-butanol and total oil concentration were calculated. The oil concentration in water product was estimated using the average response factors of the individual calibration components for the butyl ester of fatty acid and soybean oil fatty acid, but with 2-pentanol as an internal standard. The same GC method as the oil product analysis was used for the water product analysis.

### Materials

Chemicals were purchased from Sigma Aldrich unless otherwise specified. High quality water is purified water purchased from Fluka CHemie GmbH of the Sigma-Aldrich family.

Production of the Butyl Ester of Fatty Acid from Soybean Oil Fatty Acid

The butyl ester of fatty acid from soybean oil fatty acid was prepared from a formula comprising 27.3 weight percent deionized water, 0.55 weight percent 4-Morpholineethanesulfonic acid hydrate, 54.52 weight percent soybean oil fatty acid (Emery® 610 purchased from the Emery Oleochemicals LLC, Cincinnati, Ohio), 16.36 weight percent isobutanol, 1.29 weight percent 1N sodium hydroxide solution and 273 ppm of Lipolase 100 L (L0777 from Sigma Aldrich, made by Novozymes; analyzed to have 28.1 mg of protein per gram of solution). The deionized water and 4-Morpholineethanesulfonic acid hydrate buffer were mixed together and caustic added to adjust pH to 5.5. The soybean oil fatty acid was added and the two phase reaction mixture was agitated vigorously and the temperature adjusted to 30° C. The enzyme and half of the isobutanol was added and the reaction was held for 7 hours. The other half of the isobutanol was added and the reaction mixture was held for another 41 hours. The reaction mass was heated to 80° C. for 15 minutes, cooled to room temperature and allowed to phase separate without agitation. The organic layer was recovered and washed 3 times with 11 kg of water per wash. The product was analyzed by gas chromatography and found to be a mix of 92.2 weight percent butyl ester of fatty acid and 7.8 weight percent soybean oil fatty acids.

### ASPEN Modeling

The processes described herein may be demonstrated using computational modeling such as Aspen modeling (see, e.g., U.S. Pat. No. 7,666,282). For example, the commercial modeling software Aspen Plus® (Aspen Technology, Inc., Burlington, Mass.) may be used in conjunction with physical property databases such as DIPPR, available from American Institute of Chemical Engineers, Inc. (New York, N.Y.) to develop an Aspen model for an integrated butanol fermentation, purification, and water management process. This process modeling can perform many fundamental engineering calculations, for example, mass and energy balances, vapor/liquid equilibrium, and reaction rate computations. In order to generate an Aspen model, information input may include, for example, experimental data, water content and composition of feedstock, temperature for mash cooking and flashing, saccharification conditions (e.g., enzyme feed, starch conversion, temperature, pressure), fermentation conditions (e.g., microorganism feed, glucose conversion, temperature, pressure), liquid-liquid equilibrium, degassing conditions, solvent columns, pre-flash columns, condensers, evaporators, centrifuges, etc.

### Example 1—Comparative

### Hydrolysis of the Butyl Ester of Fatty Acid Using Water at High Temperature and High Pressure Using a Low Oil to Water Ratio and 1 Stage

A schematic of the reactor used to hydrolyze the butyl ester of fatty acid is shown in FIG. 1, which is described above. The butyl ester of fatty acid was used as the oil feed. High quality water was used as the water feed. The feeds were preheated to reaction conditions (260° C., 800 psig) using preheaters located in the feed lines just prior to the feed addition ports to the reactor. The water was fed at 12.5 g/min throughout the experiment. The butyl ester of fatty acid was fed at 12.5 g/min throughout the experiment. The feeds were fed counter-currently with the oil feed entering the bottom of the reactor and the water feed entering the top of the reactor. The measured volume of the reaction zone was 3.0 liter. The residence time of the oil in the reactor was estimated to be 2.0 hours. The mass ratio of oil to water was 1.0. The oil phase leaving the top of the reactor was analyzed by gas chromatography and shown to yield an overall butyl ester of fatty acid conversion of 55.0 percent.

### Example 2

### Hydrolysis of the Butyl Ester of Fatty Acid Using Water at High Temperature and High Pressure Using a High Oil to Water Ratio and 3 Stages

A schematic of the reactor used to hydrolyze the butyl ester of fatty acid is shown in FIG. 1. The butyl ester of fatty acid was used as the oil feed. High quality water was used as the water feed. The feeds were preheated to reaction conditions (260° C., 800 psig) using preheaters located in the feed lines just prior to the feed addition ports to the reactor. The water was fed at 8.9 g/min throughout the experiment. The butyl ester of fatty acid was fed at 51.6 g/min throughout the experiment. The feeds were fed counter-currently with the oil feed entering the bottom of the reactor and the water feed entering the top of the reactor. The measured volume of the reaction zone was 3.0 liter. The residence time of the oil in the reactor was estimated to be 0.5 hours. The mass ratio of oil to water was 5.8:1. The oil phase leaving the top of the reactor was analyzed by gas chromatography and shown to contain 60.1 weight percent butyl ester of fatty acid, 24.4 weight percent soybean oil fatty acid, 10.4 weight percent water and 5.0 weight percent isobutanol. The conversion of the butyl ester of fatty acid was 22.9 percent for this reaction.

An oil stream of the effluent composition sans water and isobutanol, 71.1 weight percent butyl ester of fatty acid and 29.9 weight percent soybean oil fatty acid, was subsequently fed again to the reactor at 49.9 g/min of oil flow and 9.3 g/min of water flow. The residence time of the oil in the reactor was estimated to be 0.5 hours. The mass ratio of oil to water was 5.4.The oil phase leaving the top of the reactor was analyzed by gas chromatography and shown to be 40.1 weight percent butyl ester of fatty acid, 41.6 weight percent soybean oil fatty acid, 13.0 weight percent water and 5.0 weight percent isobutanol. The conversion of the butyl ester of fatty acid was 31.0 percent.

An oil stream of the effluent composition sans water and isobutanol, 49.0 weight percent butyl ester of fatty acid and 51.0 weight percent soybean oil fatty acid, was subsequently fed again to the reactor at 49.9 g/min of oil flow and 9.3 g/min of water flow. The residence time of the oil in the reactor was estimated to be 0.5 hours. The mass ratio of oil to water was 5.4. The oil stream leaving the top of the reactor was analyzed by gas chromatography and shown to be 29.1 weight percent butyl ester of fatty acid, 56.4 weight percent soybean oil fatty acid, 10.6 weight percent water and 3.7 weight percent isobutanol. The conversion of the butyl ester of fatty acid was 30.7 percent for this step.

The overall conversion of the butyl ester of fatty acid over the three reactions was 63.3%. This occurred over 3 reactors with an average oil to water mass ratio of 5.5/1. Over the 3 reactors the ratio of oil flow to total water flow was 1.9/1.0, which is calculated from the single oil flow and net water flow for the 3 reactors.

Comparing with the Comparative Example

Comparing results in Example 2 and Comparative Example 1 shows the increased productivity of the 3 reactor set where a shorter overall residence time of 1.5 hours versus 2.0 hours and a higher oil to water ratio of 1.9 to 1.0 versus 1.0 to 1.0 yields higher conversion of butyl ester of fatty ester to the fatty acid form. These differences manifest themselves in several economically important ways. The lesser residence time implies a lesser reactor volume required to achieve the same conversion. This would reduce the capital cost of the process. The higher oil to water ratio implies less energy is required to evaporate the water and isobutanol from the butyl ester of fatty acid and fatty acid solution. This would reduce the energy required to purify the butanol and thus reduce the operating cost of the process.

### Example 3

### Hydrolysis of the Butyl Ester of Fatty Acid Using Water at High Temperature and High Pressure Using a High Oil to Water Ratio and 1 Stage

A schematic of the reactor used to hydrolyze the butyl ester of fatty acid is shown in FIG. 1. The butyl ester of fatty acid was used as the oil feed. High quality water was used as the water feed. The feeds were preheated to reaction conditions (260° C., 800 psig) using preheaters located in the feed lines just prior to the feed addition ports to the reactor. The water was fed at 1.8 g/min throughout the experiment. The butyl ester of fatty acid was fed at 12.9 g/min throughout the experiment. The feeds were fed counter-currently with the oil feed entering the bottom of the reactor and the water feed entering the top of the reactor. The measured volume of the reaction zone was 3.0 liter. The residence time of the oil in the reactor was estimated to be 2.0 hours. The mass ratio of oil to water was 7.2:1.0. The oil phase leaving the reactor was analyzed by gas chromatography and the conversion of butyl ester of fatty acid to fatty acid was 52%.

Comparing with the Comparative Example

Comparing Example 3 and Comparative Example 1 shows increasing the oil to water ratio from 1.0:1.0 to 7.8:1.0 had minimal impact on overall conversion of the ester. This difference manifest itself in reducing the amount of water to be evaporated to purify isobutanol, thereby reducing the energy required to purify butanol, thereby reducing the operating cost of the process.

### Example 4

### Recycling the Product Water Phase

A schematic of the reactor used to hydrolyze the butyl ester of fatty acid is shown in FIG. 1. The butyl ester of fatty acid was used as the oil feed. High quality water doped with isobutanol at a concentration of 1.2 weight percent was used as the water feed to simulate the recycle of the water effluent stream from the bottom of the reactor. The feeds were preheated to reaction conditions (260° C., 800 psig) using preheaters located in the feed lines just prior to the feed addition ports to the reactor. The water was fed at 12.9 g/min throughout the experiment. The butyl ester of fatty acid was fed at 13.2 g/min throughout the experiment. The feeds were fed counter-currently with the oil feed entering the bottom of the reactor and the water feed entering the top of the reactor. The measured volume of the reaction zone was 3.0 liter. The residence time of the oil in the reactor was estimated to be 2.0 hours. The mass ratio of oil to water was 1.0. The oil phase leaving the top of the reactor was analyzed by gas chromatography and shown to yield an overall butyl ester of fatty acid conversion of 54.0%.

Comparing with the Comparative Example

Comparing Example 4 with Comparative Example 1 shows that the water leaving the bottom of the reactor can be recycled back to the process with minimal impact on the conversion over the reactor. This reduces the energy required to purify the isobutanol by not having to evaporate the water from the recycle stream. This will reduce the operating cost of the process.

### Example 5

The experimental data shown in Table 2 was generated by high temperature hydrolysis of the butyl ester of fatty acid. The 100 ml stainless steel Autoclave EZE Seal® reactor (Parker Autoclave Engineers research Tools Products; Erie, Pa.) was operated at ca. 1000 psig with a maximum operating pressure of 1145 psig. The 100 mL reactor has a purged electric furnace. A 50 mL HOKE cylinder (CIRCOR Instrumentation; Spartanburg, S.C.) was used to deliver water to the reactor. The raw materials used in the experiment were sourced as described in Table 1.

TABLE 1
Source of materials used in Example 5

| Chemical | Supplier | Grade |
|---|---|---|
| Fatty Acid Butyl Ester | Produced internally | 90% FABE, |
| (FABE) | | 10% SOFA |
| Soya Oil Fatty Acid | Emery Oleochemicals | 100% |
| (SOFA) | (Cincinnatti, OH) | |
| Water | EMD Millipore (Billerica, MA) | HPLC |
| Isobutanol | Sigma Aldrich (St. Louis, MO) | >99% |
| Nitrogen | Air Liquide (Paris, France) | Ultra High |
| | | Purity |
| Magnesium Hydroxide | Sigma Aldrich | |

The butyl ester of fatty acid was transferred to the reactor via syringe at atmospheric pressure. An amount of butyl ester was added so that with expansion upon heating there is still enough head space in the reactor. The reactor contents were mixed by an air-driven overhead stirrer. An electric furnace was used to heat the reactor to operating temperature. Prior to heating, the reactor was pressure cycled with nitrogen to remove air. During heating, the reactor was closed. High pressure nitrogen was introduced into the reactor headspace after the reactor reaches operating temperature. The nitrogen feed was closed and water was added into the liquid phase of the reactor through a HOKE cylinder pressurized above reactor pressure; the relatively low quantity of water prevented it from significantly lowering the reactor temperature. Pressure in the reactor prevented liquid water from becoming gaseous. Calculations were done to ensure headspace in the reactor even after the addition of water (due to the decrease in density). The HOKE cylinder was closed after water addition and nitrogen was once again fed into the reactor headspace to maintain constant pressure. The reaction began once the water was added and afterwards samples were collected from the sample port. Pressure loss during sampling was compensated for by the headspace nitrogen feed. After each experiment the reactor was cooled (<60° C.), disassembled and the reaction contents were removed.

TABLE 2
Weight fraction of the butyl ester of fatty acid remaining in the oil
phase of the reaction mass as a function of time for four different
experimental conditions (210° C. with and without catalyst;
260° C. with and without catalyst).

| Temperature (° C.) | Time (hr) | Without Catalyst | With Catalyst |
|---|---|---|---|
| 210 | 0 | 0.91 | 0.91 |
| | 0.2 | 0.90 | 0.78 |
| | 0.4 | 0.89 | 0.72 |
| | 0.75 | 0.79 | 0.59 |
| | 1.00 | 0.74 | 0.55 |
| | 1.50 | 0.65 | 0.53 |
| | 2.00 | 0.58 | 0.54 |
| 260 | 0 | 0.91 | 0.91 |
| | 0.2 | 0.87 | 0.55 |
| | 0.4 | 0.76 | 0.52 |
| | 0.75 | 0.62 | 0.53 |
| | 1.00 | 0.56 | 0.51 |
| | 1.50 | 0.57 | 0.51 |
| | 2.00 | 0.56 | 0.51 |

### Example 6

The data in Table 3 was generated in a similar manner as to Example 5 except that after the reaction lined out, i.e. after the water hydrolysis of the butyl ester of fatty acid to fatty acid and isobutanol slowed significantly, the reactor was cooled, emptied and the collected reaction mass heated to distill any water and isobutanol present in the reaction mass from the oil phase. The oil phase and additional water was then added back to the reactor as in Example 5 and the reaction mass heated and held at temperature until the conversion again lined out. The process was repeated a third time. The reaction time was reported in sequential fashion as though the reactions occurred as a series of reactions. A mathematical model of the experimental data was developed and the constants to the model fit such that the predicted conversion reasonably matched the experimental data.

TABLE 3
Weight fraction of the butyl ester of fatty acid remaining in the oil
phase of the reaction mass after each reaction and vacuum flask
separation. Model predicted values are also included.

Weight % FABE in Solution

| | Experimental | Model Predicted |
|---|---|---|
| First reaction time (hr) | | |
| 0.0 | 0.89 | 0.89 |
| 1.0 | 0.52 | 0.48 |
| 1.2 | 0.49 | 0.47 |
| Second reaction time (hr) | | |
| 1.3 | 0.51 | 0.46 |
| 1.8 | 0.20 | 0.29 |
| 2.1 | 0.28 | 0.27 |
| Third reaction time (hr) | | |
| 2.1 | 0.28 | 0.27 |
| 2.6 | 0.18 | 0.17 |
| 2.8 | 0.17 | 0.16 |

### Example 7

The mathematical model developed in Example 6 was employed in an Aspen Plus Simulation of a 3 reactor in series reactor set with a single vacuum flash column as shown in FIG. 2 and described above, employing 260° C. across the 3 reactors in series, with reactors of adequate size to provide adequate residence time to achieve the conversions of isobutyl ester of fatty acid and water to fatty acid and isobutanol as detailed herein. The data shows the mass fractions of the four components corn oil fatty acid, butyl ester of corn oil fatty acid, water and isobutanol in each stream, the flow of the total stream, and the temperature and pressure of each stream.

In the Aspen Model, Stream 11 is a mix of aqueous and oil phases feeding the first reactor in the series of three reactors operating at 260° C. and pressure high enough to maintain the reaction mass as a liquid, estimated to be 735 psig. The mix of oil and water in Stream 11 is 86 weight % oil and 14 weight % water with the oil being 90 weight % butyl ester of fatty acid. 2.1 tonnes per hour of additional steam, Stream 29, is injected to the 32.4 tonnes per hour of reactor feed in the reactor to maintain temperature over its length. The reactor effluent, Stream 12, is fed to the top of a staged flash column where most of the water and isobutanol in Stream 12 is flashed. The flash column is built such that the remaining liquid, Stream 13, which is now 43 weight % butyl ester of fatty acid and 57 weight % fatty acid is mixed again with water at 4.5 tonnes per hour, Stream 14, and the resulting stream, Stream 15, is reheated to or near to reaction temperature, Stream 15 is fed at 30.7 tonnes per hour to Reactor 2. Steam, Stream 30, is again injected into Reactor 2 at 0.6 tonnes per hour to maintain temperature. The reactor effluent, Stream 16, is fed to the staged flash column at a tray below that for the effluent leaving Reactor 1, where most of the isobutanol and water is flashed from the stream and the remaining liquid, which is now Stream 17 comprising 27 weight % butyl ester of fatty acid and 73 weight % fatty acid is mixed with water, Stream 18, at 4.4 tonnes per hour, and the resulting stream, Stream 19, is reheated to or near to reaction temperature and fed at 29.8 tonnes per hour to Reactor 3. Additional steam, Stream 31, is injected to Reactor 3 to maintain temperature. The Reactor 3 effluent is fed to the staged flash column at a tray below the effluent leaving Reactor 2, where most of the isobutanol and water is flashed from the stream and the remaining liquid, Stream 21, comprising 18 weight % butyl ester of fatty acid and 82 weight % fatty acid at 25 tonnes per hour is returned to the fermentation process for use again as needed. The energy in Stream 21 can be used to heat other streams in the process to which it is returned. The vapor leaving the staged flash column, Stream 22, is roughly 72 weight % water and 28 weight % isobutanol, and this vapor stream is condensed and separated to further purify the isobutanol. The energy in Stream 22 can be exchanged elsewhere in the process to better energy integrate the overall process. In certain embodiments, the pressure of the staged flash column is adjusted to set the temperature of Streams 21 and 22 to allow for maximum energy integration with the rest of the process. The net effect of the process is to convert 28 tonnes per hour of a 90 weight % butyl ester of fatty acid, 10 weight % fatty acid oil stream entering the beer column into 25 tonnes per hour of an 18 weight % butyl ester of fatty acid, 82 weight % fatty acid oil stream and 4.4 tonnes per hour of isobutanol. The fatty acid can be reused in the fermentation process to capture additional isobutanol.

TABLE 4
Stream data for the 3 reactors in series at 260° C. with a
single staged flashed column. COFA = corn oil fatty acid;
iBuOH = isobutanol; T = temperature; P = pressure.

| | | | | Mass fraction | | | | |
|---|---|---|---|---|---|---|---|---|
| | | | | | isobutyl | | | |
| | T | P | | | ester of | | | |
| Stream | (° C.) | (psig) | Mass flow (kg/hr) | COFA | COFA | Water | iBuOH | Others |
| 1 | 75 | 29 | 84802 | 0.031 | 0.299 | 0.605 | 0.017 | 0.048 |
| 2 | 80 | 7 | 6955 | 0.000 | 0.000 | 0.770 | 0.204 | 0.026 |
| 3 | 87 | 9 | 77846 | 0.034 | 0.326 | 0.590 | 0.000 | 0.050 |
| 4 | 87 | 15 | 28046 | 0.094 | 0.899 | 0.005 | 0.000 | 0.002 |
| 5 | 87 | 15 | 48662 | 0.000 | 0.000 | 0.923 | 0.000 | 0.077 |
| 6 | 87 | 15 | 1138 | 0.000 | 0.000 | 0.923 | 0.000 | 0.077 |
| 7 | 87 | 15 | 45645 | 0.000 | 0.000 | 0.923 | 0.000 | 0.077 |
| 8 | 87 | 15 | 3017 | 0.000 | 0.000 | 0.923 | 0.000 | 0.077 |
| 9 | 188 | 735 | 32362 | 0.082 | 0.779 | 0.129 | 0.006 | 0.004 |
| 10 | 267 | 773 | 4316 | 0.000 | 0.000 | 0.934 | 0.044 | 0.022 |
| 11 | 260 | 735 | 32362 | 0.082 | 0.779 | 0.129 | 0.006 | 0.004 |
| 12 | 260 | 735 | 34473 | 0.421 | 0.317 | 0.153 | 0.104 | 0.005 |
| 13 | 158 | 44 | 26212 | 0.554 | 0.416 | 0.011 | 0.013 | 0.006 |
| 14 | 267 | 773 | 4475 | 0.000 | 0.000 | 0.934 | 0.044 | 0.022 |
| 15 | 260 | 735 | 30687 | 0.474 | 0.356 | 0.146 | 0.017 | 0.007 |
| 16 | 260 | 735 | 31312 | 0.577 | 0.213 | 0.154 | 0.049 | 0.007 |
| 17 | 167 | 44 | 25421 | 0.710 | 0.262 | 0.013 | 0.007 | 0.008 |
| 18 | 267 | 773 | 4389 | 0.000 | 0.000 | 0.934 | 0.044 | 0.022 |
| 19 | 260 | 735 | 29810 | 0.606 | 0.223 | 0.149 | 0.012 | 0.010 |
| 20 | 260 | 735 | 30141 | 0.659 | 0.148 | 0.153 | 0.030 | 0.010 |
| 21 | 172 | 44 | 25051 | 0.793 | 0.178 | 0.014 | 0.005 | 0.010 |
| 22 | 158 | 44 | 19242 | 0.001 | 0.002 | 0.713 | 0.279 | 0.005 |
| 23 | 102 | 22 | 107 | 0.197 | 0.422 | 0.063 | 0.312 | 0.006 |
| 24 | 102 | 22 | 7420 | 0.000 | 0.000 | 0.381 | 0.617 | 0.002 |
| 25 | 102 | 22 | 11715 | 0.000 | 0.000 | 0.930 | 0.064 | 0.006 |
| 26 | 101 | 22 | 1 | 0.000 | 0.000 | 0.335 | 0.664 | 0.001 |
| 27 | 101 | 22 | 5904 | 0.000 | 0.000 | 0.245 | 0.752 | 0.003 |
| 28 | 101 | 22 | 1515 | 0.000 | 0.000 | 0.910 | 0.089 | 0.001 |
| 29 | 267 | 773 | 2111 | 0.000 | 0.000 | 0.894 | 0.101 | 0.005 |
| 30 | 267 | 773 | 626 | 0.000 | 0.000 | 0.894 | 0.101 | 0.005 |
| 31 | 267 | 773 | 330 | 0.000 | 0.000 | 0.894 | 0.101 | 0.005 |
| 32 | 102 | 22 | 13230 | 0.000 | 0.000 | 0.928 | 0.067 | 0.005 |

What is claimed is:

1. A process for recovering alcohol from one or more fatty acid alcohol esters and/or one or more diacid alcohol esters comprising;
a) contacting one or more fatty acid and/or diacid alcohol esters in an oil phase with water producing a first reaction mixture in a first reactor at a temperature between 150° C. and 350° C. and a pressure high enough to keep the reaction in the liquid phase, using a mass ratio of oil to water that is at least about 1:1, wherein a portion of the alcohol is released from the fatty acid and/or diacid ester;
b) separating, by boiling point difference, a first alcohol and steam stream from the first reaction mixture in the first reactor, leaving a first process oil phase containing remaining fatty acid and/or diacid alcohol esters;
c) passing the first process oil phase containing remaining fatty acid and/or diacid alcohol esters to a second reactor and contacting the first process oil phase with water producing a second reaction mixture at a temperature between 150° C. and 350° C. and a pressure high enough to keep the reaction in the liquid phase, using a mass ratio of oil to water that is at least about 1:1, wherein a portion of the alcohol is released from the fatty acid and/or diacid alcohol ester;
d) separating by boiling point difference a second alcohol and steam stream from the second reaction mass in the second reactor, leaving a second process oil phase containing remaining fatty acid and/or diacid alcohol esters; and
e) recovering alcohol from the first and second alcohol and steam streams.

2. The process of claim 1 wherein steps (c) and (d) are repeated one or more times by passing each successive process oil phase to one or more an additional reactors, and alcohol is recovered from the alcohol and steam streams from all reactors.

3. The process of claim 1, wherein separation of steps (b) and (d) is by distillation or flashing.

4. The process of claim 3, wherein the separation of steps (b) and (d) is by flashing, and wherein the flashing occurs in a single staged flash column.

5. The process of claim 1, wherein the temperature is at least about 190° C. and the pressure is at least about 210 psig (1.4 megapascal).

6. The process of claim 1, wherein a water stream from at least one of the reactors is recycled as the contacting water in another reactor.

7. The process of claim 1, wherein the mass ratio of oil to water is greater than 2:1.

8. The process of claim 1, wherein the residence time for each reactor is less than four hours.

9. The process of claim 8, wherein the residence time for each reactor is less than two hours.

10. The process of claim 9, wherein the residence time for each reactor is less than one hour.

11. The process of claim 1, wherein the alcohol is a C2-C20 alcohol.

12. The process of claim 11, wherein the alcohol is butanol.

13. The process of claim 12, wherein the butanol is isobutanol.

14. The process of claim 11, wherein the alcohol is ethanol.

15. The process of claim 11, wherein the alcohol is a diol.

16. The process of claim 1, wherein alcohol is recovered from the alcohol and steam streams by distillation.

17. The process of claim 1, wherein less energy is required for distillation as compared to the energy required to recover alcohol in a stream from a process where the mass ratio of oil to water is less than 1:1.

18. The process of claim 1, wherein steps (a) and (b) occur simultaneously.

19. The process of claim 1, wherein a catalyst is added to the reactor.

20. The process of claim 19, wherein the catalyst is a solid acid catalyst or a water-tolerant Lewis acids catalyst.
